(19) United States
(12) Patent Application Publication
(10) Pub. No.: US 2005/0062278 A1
(43) Pub. Date: Mar. 24, 2005

(54) Fender brackets

(76) Inventors: Larry L. Griffin, Hesperia, CA (US); Earl H. Wolleson, Hesperia, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/669,983

(22) Filed: Sep. 24, 2003

(51) Int. Cl. A61K 31/70; A01N 43/04

(52) U.S. Cl. 280/847

(57) ABSTRACT

A bracket for supporting a fender of a motorcycle can be configured such that a larger tire can be accommodated. For example, the fender bracket can be shaped so as to laterally offset the fender from its original position so that a larger tire can be mounted to the motorcycle and received within the fender. The fender bracket can be included in a kit for replacing the original fender brackets of a motorcycle so as to offset the fender from its original position. Additionally, the kit can include adjustment members for use with original mounting apertures provided on the motorcycle frame so as to allow the fender brackets to be adjusted.

### BACKGROUND OF THE INVENTION

1. Field of the Invention

The present application is directed to a mounting arrangement for the rear fender of a motorcycle and in particular, brackets for laterally offsetting a rear fender of a motorcycle.

2. Description of the Related Art

Large motorcycles are often the pride of their owners. These machines are especially appreciated for their distinct appearance, distinct sound, and high performance. Some owners of large motorcycles are independent, original, and creative individuals who like to oppose a personal touch to the appearance or to the engine performance of their motorcycle. However, other owners prefer to preserve the original appearance of their motorcycle. Therefore, original equipment manufacturers (OEM) as well as generic component fabricators offer large assortments of custom parts and accessories for customizing these motorcycles to satisfy a great number of different preferences.

A common modification performed by motorcycle owners is the replacement of the original rear tire with a wider tire. This modification enhances the expression of power and changes the riding characteristic of the motorcycle.

The original rear wheel tire on certain Harley-Davidson Softail™ motorcycles, including at least the model years 1987 through 1999, is a Series 130 mm (5.1 inches). The width of the frame allows for the installation of a Series 160 mm (6.3 inches) tire. However, simply installing a 160 mm tire on to the original equipment rim is not satisfactory because the 160 mm tire will rub on the original equipment rear fender of such motorcycles. In particular, because the rear wheel of Harley-Davidson Softail™, as well as many other motorcycles, is offset from the longitudinal axis of the frame of the motorcycle, a larger tire is likely to rub on one side of the inner surface of the fender.

Numerous kits are presently on the market for replacing original rear fender assembly with a larger assembly that can accommodate a larger tire as well as kits for completely replacing the swingarm assembly and/or moving the engine and transmission. However, these solutions change the appearance of the motorcycle and are difficult and costly to install.

### SUMMARY OF THE INVENTION

One aspect of at least one of the inventions disclosed herein includes the realization that a larger tire can be accommodated by the original equipment fender of certain motorcycles if the fender is shifted laterally so as to move the longitudinal center plane of the fender toward the center plane of the tire. For example, the alignment of the rear tire of many modern motorcycles relative to the frame of the motorcycle is offset laterally to provide a desired balance for the motorcycle and to compensate for torsional forces generated through the interaction of the drive train with the rear wheel. However, for aesthetic reasons, the rear fender of the motorcycle is centered along the longitudinal center plane or center axis of the frame of the motorcycle.

Accordingly, in one embodiment of at least one of the inventions disclosed herein, the fender is laterally offset from the longitudinal center plane of the frame, thereby moving the fender more into alignment with the tire. This lateral offset from the center plane of the frame is not immediately perceptible. Thus, an owner of such motorcycle can largely retain the original fender, yet install a larger tire than that which could be accommodated by the original fender assembly.

In accordance with another aspect of at least one of the inventions disclosed herein, a fender bracket kit for a motorcycle comprises first and second brackets including first and second frame mounting points, respectively, configured for mounting the brackets to a frame of a motorcycle. The first and second brackets including third and fourth mounting points for supporting a rear fender over a rear tire of the motorcycle. The first and second brackets are configured to support the fender such that a centerline of the fender is offset from a centerline of the frame.

In accordance with yet another aspect of at least one of the inventions disclosed herein, a rear fender assembly for motorcycle comprises a rear fender and a bracket assembly configured to support the rear fender above a rear wheel of a motorcycle. The bracket assembly includes at least one bracket configured to support the rear fender in the position such that a centerline of the rear fender is offset from a centerline of the frame of the motorcycle.

In accordance with a further aspect of at least one of the inventions disclosed herein, a method for increasing a maximum size of a tire that can be mounted to motorcycle having a rear fender aligned with a center plane of a frame of the motorcycle and supported by original brackets comprises removing the original brackets, and mounting the fender so as to offset the rear fender from the center plane of the frame.

### BRIEF DESCRIPTION OF THE DRAWINGS

FIG. 1 is a left-side elevational view of a motorcycle having an original equipment rear fender and bracket assembly therefor;

FIG. 2 is a top plane view of the motorcycle illustrated in FIG. 1 with the seat and certain upper frame members and the fender brackets removed;

FIG. 3 is a rear schematic elevational view of the motorcycle illustrated in FIG. 1 illustrating the rear wheel swingarm and frame assembly thereof;

FIG. 4 is a schematic rear elevational view of an improved fender bracket assembly constructed in accordance with certain aspects of the inventions disclosed herein;

FIG. 5 is a left-side elevational view of the left-side bracket illustrated in FIG. 4;

FIG. 6 is a bottom plan view of the left-side bracket illustrated in FIG. 5;

FIG. 7 is a right-side elevational view of the right-side bracket illustrated in FIG. 4.

FIG. 8 is a bottom plan view of the right-side bracket illustrated in FIG. 7;

FIG. 9 is a front elevational view of an adjusting member that can be used with the mounting brackets illustrated in FIGS. 4-8;

FIG. 10 is a side-elevational view of the adjusting member illustrated in FIG. 9;

FIG. 11 is a left-side schematic elevational view of two of the adjusting devices illustrated in FIGS. 9 and 10 used in conjunction with the left-side bracket illustrated in FIG. 5;

FIG. 12 is a further illustrative orientation of the adjusting devices illustrated in FIG. 11; and

FIG. 13 is yet another illustrative orientation of the adjusting devices illustrated in FIG. 11.

### DETAILED DESCRIPTION OF THE PREFERRED EMBODIMENT

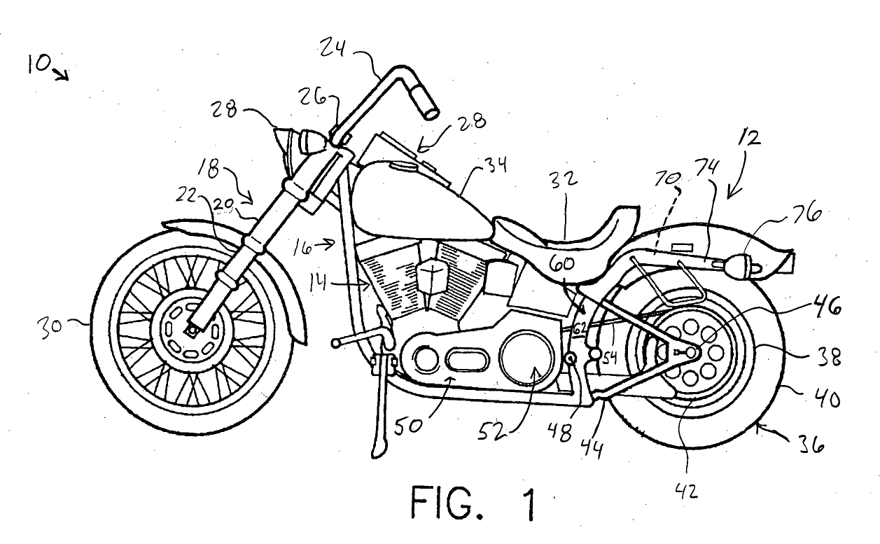

With reference to FIG. 1, a motorcycle is illustrated in side elevational view and as identified generally by the reference numeral 10. The motorcycle 10 includes a rear fender support assembly 12 that can be modified in accordance with certain features and aspects of the inventions disclosed herein. As such, the motorcycle 10 provides a typical environment in which the present inventions can be used.

The motorcycle 10 is generally comprised of an engine 14 and a frame assembly 16 supporting the engine 14. The illustrated frame assembly 16 is of a double-cradle-type frame. The frame assembly 16 supports the engine 14 and a front fork assembly 18, which is known as a “handstand-type telescopic” fork assembly. Each side of the fork assembly 18 includes an outer tube 20 and an inner tube 22.

A bracket assembly, which can include upper and lower brackets, connects the outer tube 20 of the two front forks. Additionally, the bracket assembly is pivotally supported by a head tube defined at the forward portion of the frame assembly 16.

A handlebar 24 is mounted to the bracket assembly. In particular, the handlebar 24 is mounted to the upper bracket of the bracket assembly with a clamp 26.

The handlebar 24 can carry a variety of controls. For example, the handlebar 24 can include a twist-grip-type throttle normally positioned on the right end of the handlebar 24, a front brake lever disposed adjacent to the throttle grip, a clutch lever, typically disposed adjacent the left end of handlebar 24, as well as other controls.

The bracket assembly also supports the headlight 28. The bracket assembly can also support additional gauges, such as, for example, but without limitation, a tachometer, a speedometer, a fuel gauge, a battery state indicator, an oil pressure gauge, etc. In the illustrated embodiment, the motorcycle 10 includes a gauge cluster 28 disposed rearwardly from the handlebar clamp 26 and supported by the frame assembly 16.

A wheel 30 is journaled for rotation at a lower end of the fork assembly 18. Additionally, a front brake (not shown) is also mounted to the wheel and partially supported by the lower end of the fork assembly 18.

A rider seat 32 is disposed rearwardly from the handlebar 24 and supported by a seat rail (not shown). Positioned as such, a rider can sit on the seat 32 while holding the handlebar 24.

A fuel tank 34 is supported by a tank rail (not shown) of the frame assembly 16 and is disposed forwardly from the seat 32, between the seat 32 and the handlebar 24. However, a decorative cover similar in shape to the fuel tank 34 can be installed in this position in lieu of the gas tank 34, with the gas tank located in another position.

A rear wheel 36 is journaled for rotation relative to the frame assembly 16. The rear wheel 36 includes a rim 38 having a hub at the center thereof and a tire 40 supported by the rim 38. A driven pulley 42 is rotatably mounted to the hub of the rim 38 so as to rotate therewith. As such, the driven pulley 42 can transmit a torque to the rear wheel 36.

In the illustrated embodiment, the rear wheel 36 is supported relative to the frame assembly 16 with a swingarm assembly 44. However, the rear wheel 36 can be supported relative to frame assembly 16 in any known manner.

In the illustrated embodiment, the swingarm assembly 44 is constructed in accordance with the well known Softail™ design. As such, the swingarm assembly 44 includes a mounting aperture 46 for supporting an axle for the rear wheel 36.

The swingarm assembly 44 defines a pivot axis 48 about which the assembly 44 can pivot. The spring and the shock absorber (not shown) lie generally horizontally and are supported by a lower portion of the frame assembly 16. Up and down movement of the wheel 36 causes a corresponding generally horizontal forward and rearward movement in the lower portion of the swingarm assembly 44.

The spring and the shock absorber are attached to a lower portion of the swingarm assembly 44 and thereby provide a bias and a damping force for the up and down movements of the rear wheel 36. This design is well known in the art and a further description of this assembly is not necessary for one of ordinary skill in the art to make and use the inventions disclosed herein.

The illustrated driven pulley 42 is driven by a transmission 50. A portion of the transmission 50 is contained at least partially within a crankcase transmission assembly of the engine 14. The transmission 50 drives the rear wheel 36 through a final drive assembly 52.

The final drive assembly 52 includes a drive sprocket (not shown) which is driven by a crankshaft (not shown) of the engine 14 through a plurality of gear sets (not shown) defining a speed change transmission. The final drive assembly 52 also includes the driven sprocket 42 mounted to the rear wheel 36. A flexible transmitter such as a toothed rubber belt 54 is wound around the drive sprocket and the driven sprocket 42.

The transmission 50 also includes a gear shifter (not shown) for shifting the transmission 50 between different gear ratios defined by the gear sets disposed therein. The gear shifter can be disposed adjacent to a left foot of a rider of the motorcycle 10. However, other types of shifters can be used. A brake pedal is typically disposed adjacent a rider's right foot for controlling a rear brake (not shown) for the rear tire 36.

With reference to FIG. 1, the engine 14 can be a V-twin type engine operating on a four-cycle principle. However, this is merely one exemplary type of engine that can be used as the engine 14. Other types of engines can also be used. A further description of the engine 14 is not necessary for one of ordinary skill in the art to make and use the inventions disclosed herein.

With continued reference to FIG. 1, a rear portion of the frame assembly 16 includes a swingarm support portion 60. In the illustrated embodiment, the swingarm support portion 60 comprises left and right swingarm support members 62, 64. The swingarm support portion 62 is curved and extends generally vertically, in front of the rear wheel 36.

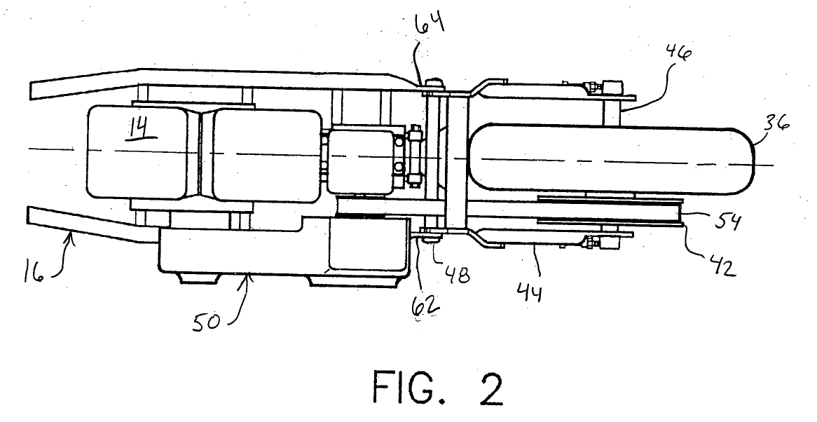

As shown on FIG. 2, the left and right swingarm support portions 62, 64 support the swingarm pivot shaft 48 so that the swingarm assembly 44 can pivot about a pivot axis defined by the swingarm pivot shaft 48.

FIG. 3 illustrates a schematic rear elevational view of a portion of the motorcycle 10 including the rear tire 36, the swingarm assembly 44, and the left and right swingarm supports 62, 64. Additionally, FIG. 3 illustrates a rear fender 66 supported above the rear tire 36. A seat 68 is connected to an upper surface of the rear fender 66. However, it is to be noted that the rear seat 68 is optional and is not included on all such motorcycles 10.

As shown on FIG. 3, left and right fender brackets 70, 72 extend upwardly from the left and right swingarm supports 62, 64. The left and right fender bracket 70, 72 are connected to the fender 66 with bolts 71, 73, respectively. The fender brackets 70, 72 include offsetting portions that extend inwardly from the bolts 71, 73, toward the fender 66. For example, the left side fender bracket 70 defines an offset Oₒ, thereby locating the fender 66 such that the centerline 79 of the fender 66 is aligned with the centerline 78 of the frame assembly 16. On some motorcycles, the fender brackets are symmetrical with each other. However, the fender brackets 70, 72 of the motocycle 10 are not symmetrical due to differences in the shapes of the swingarm support portions 62, 64.

With reference to FIG. 1, the left side fender bracket 70 is covered with an outer decorative cover 74. The cover 74 provides an enhanced aesthetic outer appearance of the fender brackets 70, 72. For example, the cover 74 can be chromed. Additionally, the cover 74 can provide a covering for electrical conduits extending to devices such as, for example, a turn indicator light 76.

With reference again to FIG. 3, the frame assembly 16 defines a longitudinal center plane 78 extending therethrough. The center plane 78 extends through a longitudinal center of the motorcycle 10, which, on a typical motorcycle 10, will bisect the seat 32, the handlebar 24, and the fork assembly 18.

FIG. 3 also illustrates a center plane 80 of the rear tire 36. As shown in FIG. 3, the center plane 80 of the tire 36 is laterally offset from the center plane 78 of the motorcycle 10 by a distance identified generally by the reference numeral 82. This lateral offset 82 is determined based on certain desired handling characteristics for the motorcycle 10. For example, a manufacturer of the motorcycle 10 takes into consideration various parameters including the torsional dynamics associated with the power transmission to and from the rear tire 36. The offset 82 illustrated in FIG. 3 is represented schematically.

The rear fender 36 of such motorcycle 10 also has its center 79 aligned along the center plane 78 of the motorcycle frame assembly 16. With the rear tire 36 offset from the center plane 78 as such, the right side of the tire 36 is closer to a right side inner surface 84 of the fender 66.

Additionally, the rear fender 66 can include stiffening ribs identified generally by the reference numeral 86, configured to enhance the rigidity of the fender 66. Thus, the inner surface 84 or the stiffening ribs 86 determine the maximum width of the tire that can be accommodated within the fender 66. If a tire having a width that is too large is installed on to the rim 38, such a tire will rub against either the right side inner surface 84 of the fender 66 or the corresponding stiffening rib 86.

The left and right swingarm supports 62, 64 include accessory mounting apertures 90, 92, respectively. The accessory apertures 90, 92 can be used for mounting various accessories to the motorcycle 10. For example, the cover 74 can be mounted over the left side fender bracket 70 using the aperture 90. The apertures 90, 92 can include internal threads to facilitate attachment of accessories thereto. A cover similar to the cover 74 can be attached over the right side fender bracket 72 using the accessory aperture 92.

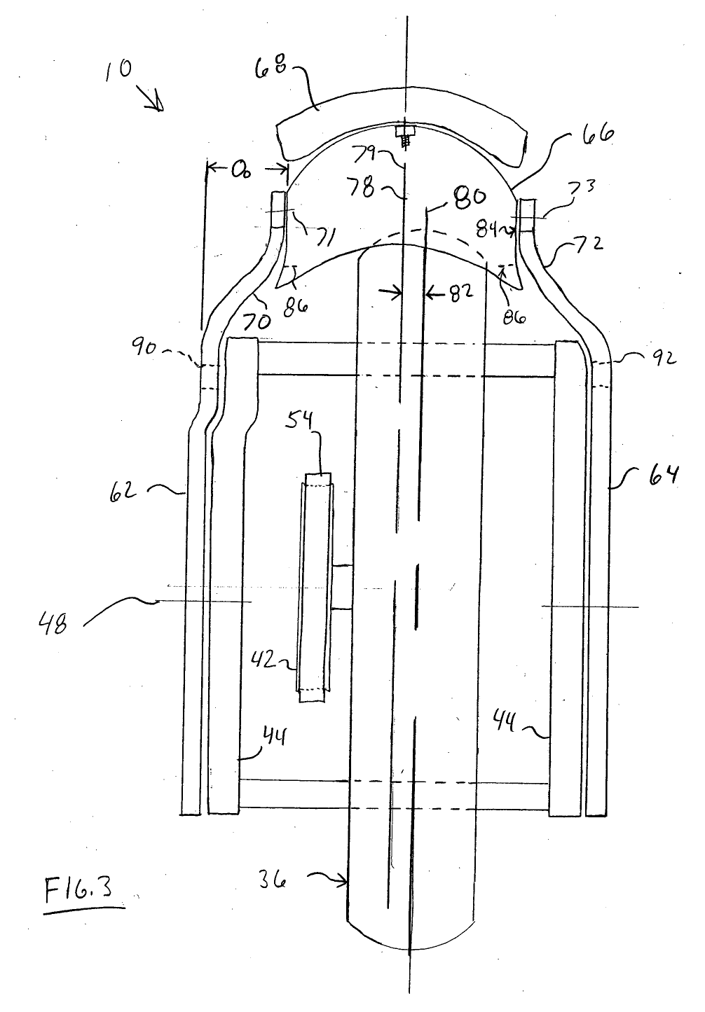

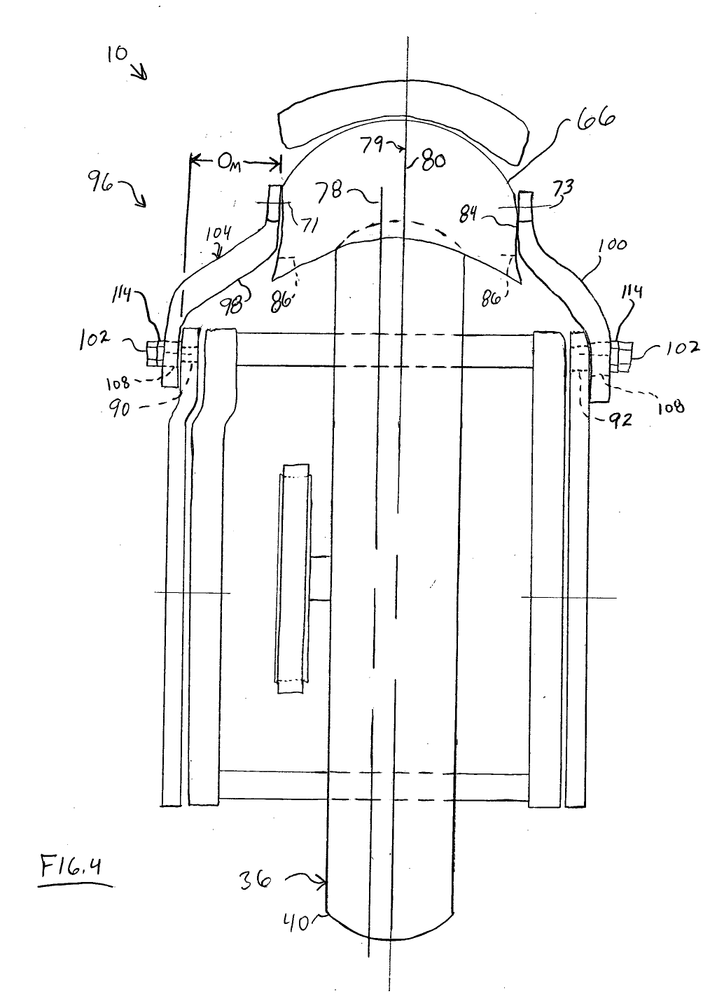

FIG. 4 illustrates, schematically, a kit 96 installed on the motorcycle 10 for laterally offsetting the fender 66 relative to a longitudinal centerline or the center plane 78 of the motorcycle 10. In the illustrated embodiment, the kit 96 includes left and right fender brackets 98, 100 mounted to the accessory apertures 90, 92, respectively. The left and right fender brackets 98, 100 are configured to mount the fender 66 so that the center line 79 of the fender 66 is positioned closer to the center line 80 of the rear tire 36.

In the illustrated embodiment, the left and right brackets 98, 100 are shaped so as to align the center line 79 of the fender 66 with the center line 80 of the rear tire 36. As such, a larger size rear tire can be used to replace the rear tire 40. On some motorcycles, where the original equipment for the motorcycle 10 included a 130 mm Series rear tire, a 160 mm rear tire can be mounted on the rim 38 without rubbing on the inner surface 84 or the stiffening ribs 86 of the rear fender 66.

In the illustrated kit 96, bolts 102 extend through apertures defined in the left and right brackets 98, 100 and into the accessory apertures 90, 92 so as to retain the left and right brackets 98, 100 in place.

FIGS. 5-8 illustrate the left and right brackets 98, 100 in greater detail. In particular, FIGS. 5 and 6, respectively, show a left-side elevational view and a bottom plan view of the left side bracket 98. FIGS. 7 and 8, respectively, show a right-side elevational view and a bottom plan view of the right side bracket 100.

With reference to FIG. 5, the left side fender bracket 98 includes a mounting portion 103, an offsetting portion 104, and a fender support portion 106. The mounting portion 103 includes a plurality of apertures 108 configured to be alignable with the accessory mounting apertures 90, 92 (FIGS. 3 and 4). Thus, as shown on FIG. 4, after the original fender mounting brackets 70, 72 have been removed from the frame 16, the apertures 108 can be aligned with the apertures 90 and secured to each other with the bolt arrangement 102.

The apertures 108 can have an inner diameter that is approximately the same as the outer diameter of the bolt 102 so as to provide a tight fit and a repeatable alignment with the aperture 90. Optionally, the apertures 108 can have an inner diameter that is greater than the outer diameter of the bolt 102. As such, the bracket 98 can be adjusted relative to the aperture 90.

With continued reference to FIGS. 5 and 6, the offsetting portion 104 extends from the mounting portion 103 to the fender support portion 106. As shown in FIG. 6, the offsetting portion defines a lateral offset from the mounting portion to the fender support portion 106. In the illustrated embodiment, the offsetting portion 104 is thicker than the mounting portion 103 in the fender support portion 106. As shown in FIGS. 4 and 6, the configuration of the offsetting portion 104 defines an offset Om which is larger than the offset Oₒ defined by the original fender bracket 70.

The fender support portion 106 also includes a plurality of apertures 110 that is configured to support the fender 66 and/or other accessories. Preferably, the apertures 110 are configured to be alignable with the original mounting apertures defined in the fender 66.

With reference to FIGS. 7 and 8, the bracket 100 is constructed similarly to the bracket 98. However, the bracket 100 includes an offsetting portion 112 that defines an offset that is smaller than the offset Oₘ defined by the bracket 98. The remaining portions of the bracket 100 can be constructed similarly or identically to the corresponding portions of the bracket 98. Thus, a further description of the bracket 100 is not necessary for one of ordinary skill in the art to make and use the inventions disclosed herein.

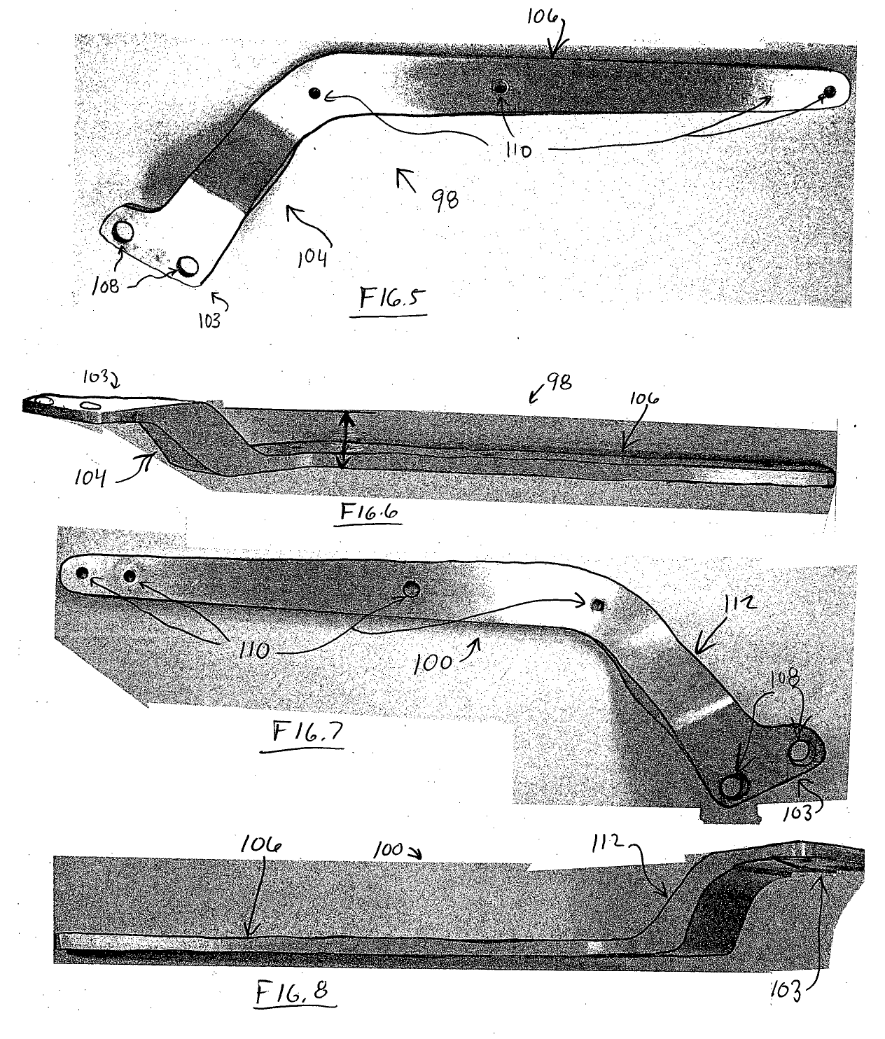

With reference to FIGS. 9-13, a further advantage is provided where the apertures 108 have an inner diameter that is larger than the outer diameter of the bolts 102, and an adjustment bushing 114 is disposed between the bolt 102 and the aperture 108.

With reference to FIGS. 9 and 10, the adjustment bushing 114 includes a head portion 116 and the sleeve portion 118. The head portion 116 can have any shape. In the illustrated embodiment, the head portion 116 defines a hexagonal outer surface that can be received by a wrench. However, the head portion 116 can have other shapes.

The sleeve portion 118 includes an outer surface configured to fit within the inner diameter of the apertures 108. The sleeve portion 118 defines an axis 121 extending generally through a center of the diameter defined by the outer surface of the sleeve portion 118.

The adjustment member 114 also includes the central aperture 122. The inner diameter of the central aperture 122 is smaller than the diameter defined by the outer surface of the sleeve portion 118. Additionally, the aperture 122 defines a center axis 124 extending through a center of the diameter defined by the inner surface of the aperture 122. As shown in FIGS. 9 and 10, the axis 122 is offset from the axis 121. As such, the adjustment member 114 can be used to adjust an orientation of the fender bracket 98.

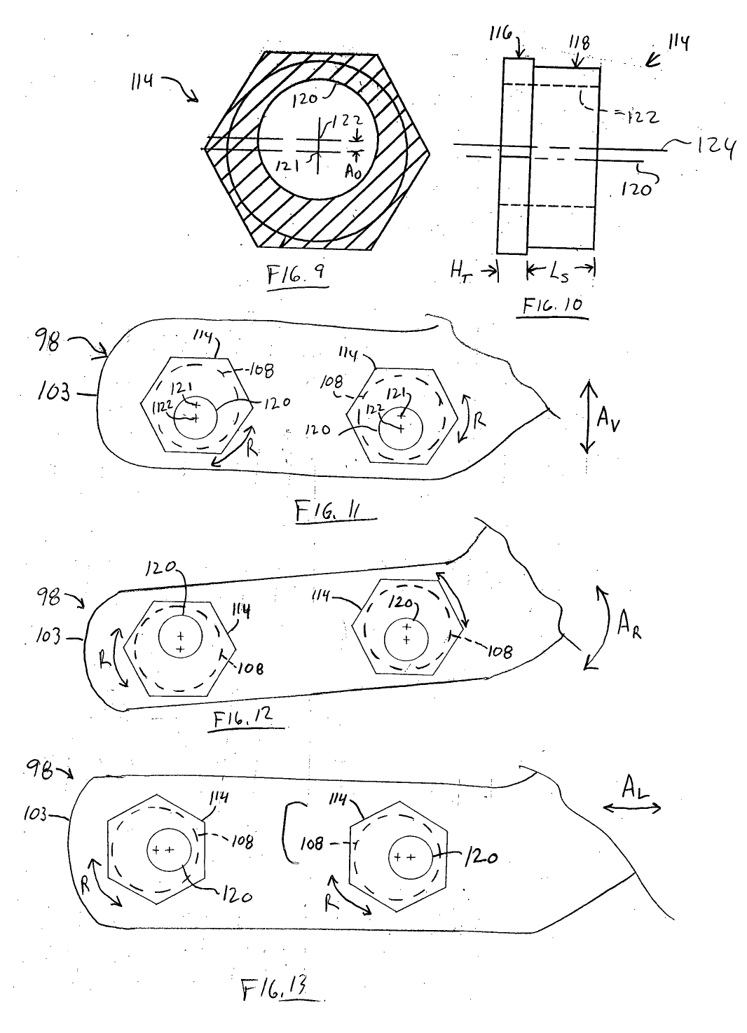

For example, with reference to FIG. 11, the mounting portion 103 and two adjusting members 114 are illustrated schematically therein. In FIG. 11, the sleeve portions 118 of each adjusting member 114 are inserted into the apertures 108 of the left side fender bracket 98. Additionally, a bolt 102 (not shown in FIG. 11), is inserted through the aperture 120 defined in the adjustment member 114 and extends into the aperture 90 of the frame 116.

When the adjustment members 114 are rotated, i.e., in the direction of arrows R, the position of the aperture 108 relative to the position of the aperture 120 can be changed. For example, as illustrated in FIG. 11, the adjusting members 114 can be rotated such that the axis 121 of the sleeve portion 118 is higher than the axis 122 of the aperture 120. Thus, the mounting portion 103 and thus the bracket 98 is adjusted vertically in the direction of arrow Aᵥ. In particular, the bracket 98 is raised vertically relative to the aperture 120, which is in direct alignment with the aperture 90. Thus, the bracket 98 is raised relative to the frame 16. As such, a user can decide to raise the fender 66 relative to the frame 16. In another orientation, not shown, the adjustment member 114 can be rotated 180° but from the position illustrated in FIG. 11, thereby lowering the bracket 98 relative to the frame 16.

With reference to FIG. 12, the adjustment members 114 can also be used to change a rotational position of the bracket 98 relative to the frame 116. For example, by rotating the adjustment members 114, the relative positions of the apertures 108 and 120 can be changed so as to adjust the position of the bracket 98 in a rotational direction, indicated generally by the arrow A_{R}. In the illustrated position, the adjustment member 114 illustrated on the right hand side of FIG. 12 is in the same position illustrated in FIG. 11. However, the adjustment member 114 on the left hand side of FIG. 12 has been rotated 180°. Thus, the left hand side of the mounting portion 103 is urged downwardly relative to the position illustrated in FIG. 11. Thus, the bracket 98 is adjusted in a counter-clockwise direction relative to the position illustrated in FIG. 11. As such, the extreme rearward end of the fender mounting portion 106 (not shown in FIG. 12) will be raised relative to the frame 16. Thus, the adjusting members 114 provide a device for allowing a user to adjust the position of the fender 66 in another manner relative to the rear tire 36.

With reference to FIG. 13, the adjustment members 114 can also be used to adjust a position of the bracket 98 in a longitudinal direction relative to the frame 16, identified by the arrow A_{L}. In the arrangement illustrated in FIG. 13, the adjustment members 114 have been rotated 90°, counter-clockwise, relative to the position illustrated in FIG. 11. Thus, the bracket 98 is positioned slightly forward and slightly lower than the position illustrated in FIG. 11. As such, the adjustment members 114 allow a user to move the fender 66 in a longitudinal direction A_{L}.

With reference again to FIG. 9, the offset A_{O} between the axis 121, 122 provides the adjustability described above with reference to FIGS. 11-13. The offset A_{O} can have any dimension. In the illustrated embodiment, the offset A_{O} is approximately 5/100 of 1″. In this illustrated embodiment, the outer diameter defined by the outer surface of the sleeve portion 118 can be approximately ¾ of 1″ and the inner diameter of the aperture 122 can be approximately ½ of 1″. The thickness of the head portion 116, identified generally by the reference letter Hₜ in FIG. 10, can be approximately ⅛ of 1″. Additionally, the length of the sleeve portion 118, identified generally by the reference letter Lₛ, can be approximately ¼ of 1″. It is to be noted that the above noted dimensions are merely an illustrative example of an adjustment member 114 that can be used with the motorcycle 10. Of course, the adjustment members 114 can be provided with other dimensions as desired.

1. A fender bracket kit for a motorcycle comprising first and second brackets including first and second frame mounting points, respectively, configured for mounting the brackets to a frame of a motorcycle, the first and second brackets including third and fourth mounting points for supporting a rear fender over a rear tire of the motorcycle, the first and second brackets being configured to support the fender such that a centerline of the fender is offset from a centerline of the frame.

2. The kit according to claim 1, wherein the first and second brackets are configured to offset the centerline of the fender at least about one half of one inch from the centerline of the frame.

3. The kit according to claim 1, wherein the first and second frame mounting points comprise apertures sized to receive fasteners.

4. The kit according to claim 3, wherein the first and second brackets are not symmetrical with each other.

5. The kit according to claim 3, wherein the first and second brackets include first and second offsetting portions, respectively, the first offsetting portion defining an offset that is larger man and offset defined by the second offsetting portion.

6. The kit according to claim 1 additionally comprising at least a first adjustment bushing, the first adjustment bushing comprising a first sleeve portion including a first outer surface defining a first axis and a first aperture extending through the first sleeve portion and defining a second axis, the first axis being offset from the second axis.

7. The kit according to claim 6, wherein the first and second axes are generally parallel to each other.

8. The kit according to claim 6, wherein the first outer surface of the first sleeve portion is configured to be received within an aperture included in the first mounting point.

9. A rear fender assembly for motorcycle comprising a rear fender, a bracket assembly configured to support the rear fender above a rear wheel of a motorcycle, the bracket assembly including at least one bracket configured to support the rear fender in the position such that a centerline of the rear fender is offset from a centerline of the frame of the motorcycle.

10. The rear fender assembly according to claim 9 additionally comprising a second bracket configured to cooperate with the first bracket so as to support the rear fender.

11. A kit for changing the location of a rear fender of a motorcycle comprising means for laterally offsetting a rear fender of a motorcycle.

12. The kit according to claim 11, wherein the means for laterally offsetting comprises means for centering the rear fender over a rear tire of the motorcycle.

13. A method for increasing a maximum size of a tire that can be mounted to motorcycle having a rear fender aligned with a center plane of a frame of the motorcycle and supported by original brackets comprising removing the original brackets, and mounting the fender so as to offset the rear fender from the center plane of the frame.

14. The method according to claim 13, wherein the step of offsetting comprises moving the rear fender such that a center plane of the rear fender is moved away from the center plane of the frame.

15. The method according to claim 13 additionally comprising mounting the rear fender to the frame with a bracket.
